(19) United States
(12) Patent Application Publication
(10) Pub. No.: US 2009/0076559 A1
(43) Pub. Date: Mar. 19, 2009

(54) Adherent Device for Cardiac Rhythm Management

(75) Inventors: Imad Libbus, Saint Paul, MN (US); Badri Amurthur, Los Gatos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corventis, Inc., San Jose, CA (US)

(21) Appl. No.: 12/209,262

(22) Filed: Sep. 12, 2008

Related U.S. Application Data
(63) Continuation of application No. 60/972,537, filed on Sep. 14, 2007.
Application No. 60/972,537 is a continuation of application No. 60/972,616, filed on Sep. 14, 2007.
Application No. 60/972,616 is a continuation of application No. 61/047,875, filed on Apr. 25, 2008.
Application No. 61/047,875 is a continuation of application No. 61/055,666, filed on May 23, 2008.

(51) Int. Cl. A61N 1/39

(52) U.S. Cl. 607/6

(57) ABSTRACT

An adherent device to monitor and treat a patient comprises an adhesive patch to adhere to a skin of the patient. At least two electrodes are connected to the patch and capable of electrically coupling to the patient. Sensor circuitry is coupled to the at least two electrodes and configured to measure at least two of an electrocardiogram signal of the patient, a respiration signal of the patient or an activity signal of the patient. Therapy circuitry is coupled to the at least two electrodes and configured to deliver a high-energy shock therapy for cardioversion and/or defibrillation. A processor system comprising a tangible medium and coupled to the sensor circuitry and therapy circuitry, the processor is configured to generate a treatment signal to deliver the high-energy shock therapy in response to the at least two of the electrocardiogram signal, the respiration signal or the activity signal.

### CROSS-REFERENCES TO RELATED APPLICATIONS

The present application claims the benefit under 35 USC 119(e) of U.S. Provisional Application Nos. 60/972,616 and 60/972,537 both filed Sep. 14, 2007, 61/047,875 filed Apr. 25, 2008, and 61/055,666 filed May 23, 2008; the full disclosures of which are incorporated herein by reference in their entirety.

The subject matter of the present application is related to the following applications: 60/972,512; 60/972,329; 60/972,354; 60/972,363; 60/972,343; 60/972,581; 60/972,629; 60/972,316; 60/972,333; 60/972,359; 60/972,336; 60/972,340 all of which were filed on Sep. 14, 2007; 61/046,196 filed Apr. 18, 2008; 61/055,645, 61/055,656, 61/055,662 all filed May 23, 2008; and 61/079,746 filed Jul. 10, 2008.

The following applications are being filed concurrently with the present application, on Sep. 12, 2008: Attorney Docket Nos. 026843-000110US entitled “Multi-Sensor Patient Monitor to Detect Impending Cardiac Decompensation Prediction”; 026843-000220US entitled “Adherent Device with Multiple Physiological Sensors”; 026843-000410US entitled “Injectable Device for Physiological Monitoring”; 026843-000510US entitled “Delivery System for Injectable Physiological Monitoring System”; 026843-000710US entitled “Adherent Device for Respiratory Monitoring”; 026843-000810US entitled “Adherent Athletic Monitor”; 026843-000910US entitled “Adherent Emergency Monitor”; 026843-001320US entitled “Adherent Device with Physiological Sensors”; 026843-001410US entitled “Medical Device Automatic Start-up upon Contact to Patient Tissue”; 026843-001900US entitled “System and Methods for Wireless Body Fluid Monitoring”; 026843-002010US entitled “Adherent Cardiac Monitor with Advanced Sensing Capabilities”; 026843-002410US entitled “Adherent Device for Sleep Disordered Breathing”; 026843-002710US entitled “Dynamic Pairing of Patients to Data Collection Gateways”; 026843-003010US entitled “Adherent Multi-Sensor Device with Implantable Device Communications Capabilities”; 026843-003110US entitled “Data Collection in a Multi-Sensor Patient Monitor”; 026843-003210US entitled “Adherent Multi-Sensor Device with Empathic Monitoring”; 026843-003310US entitled “Energy Management for Adherent Patient Monitor”; and 026843-003410US entitled “Tracking and Security for Adherent Patient Monitor.”

### BACKGROUND OF THE INVENTION

1. Field of the Invention

The present invention relates to patient monitoring and therapy. Although embodiments make specific reference to patient monitoring and therapy with an adherent patch, the system methods and device described herein may be applicable to many applications in which physiological monitoring and therapy are used, for example wireless physiological monitoring for extended periods.

Patients are often treated for diseases and/or conditions associated with a compromised status of the patient, for example a compromised physiologic status. In some instances a patient may have suffered a heart attack and require treatment and/or monitoring after release from the hospital. Although implantable devices such as pacemakers can provide effective treatment in some instances, implantable devices are invasive and may not be suitable for some patients.

Work in relation to embodiments of the present invention suggests that known methods and apparatus for long term monitoring and treatment of patients may be less than ideal. In some instances, a patient may require monitoring to determine whether the patient actually needs an implantable device and still be at risk for a heart attack while being monitored. With patients who are known to need an implantable device, for example a pacemaker, at least some patients may not be treated immediately, and these patients could benefit from an interim device that could provide treatment, if needed. In some instances, the device may be worn by the patient for an extended period, for example at least one week. Work in relation to embodiments of the present invention suggests that current monitoring and/or therapeutic devices that are worn by the patient may be somewhat uncomfortable, which may lead to patients not wearing the devices, such that data collected may be less than ideal. Also, therapeutic devices that are removed by the patient may not be capable of providing therapy after removal.

Work in relation to embodiments of the present invention also suggests that current wearable therapeutic devices may have a less than ideal sensitivity and specificity with respect to the detection of conditions requiring intervention. As intervention with a wearable device may use high energy shocks and/or voltages for therapy, it would be helpful such devices delivered therapy with fewer false positives.

Although implantable devices may be used in some instances, many of these devices can be invasive and/or costly, and may suffer at least some of the shortcomings of known wearable devices.

Therefore, a need exists for improved patient monitoring and therapy. Ideally, such improved patient monitoring would avoid at least some of the short-comings of the present methods and devices.

2. Description of the Background Art

The following U.S. patents and Publications may describe relevant background art: U.S. Pat. Nos. 4,121,573; 4,955,381; 4,981,139; 5,080,099; 5,353,793; 5,511,553; 5,544,661; 5,558,638; 5,724,025; 5,772,586; 5,862,802; 6,047,203; 6,117,077; 6,129,744; 6,225,901; 6,385,473; 6,416,471; 6,454,707; 6,527,711; 6,527,729; 6,551,252; 6,595,927; 6,595,929; 6,605,038; 6,645,153; 6,821,249; 6,980,851; 7,020,508; 7,054,679; 7,153,262; 2003/0092975; 2003/0212319; 2005/0113703; 2005/0131288; 2006/0010090; 2006/0031102; 2006/0089679; 2006/0155183; 2006/0161205; 2006/122474; 2006/0224051; 2006/0264730; 2007/0021678; 2007/0038038; 2007/0073361; and 2007/0150008.

### BRIEF SUMMARY OF THE INVENTION

The present invention relates to patient monitoring. Although embodiments make specific reference to monitoring and therapy an adherent patch, the system methods and device described herein may be applicable to many applications in which physiological monitoring and therapy are used, for example wireless physiological monitoring for extended periods.

In a first aspect, embodiments of the present invention provide an adherent device to monitor and treat a patient. The device comprises an adhesive patch to adhere to a skin of the patient. At least two electrodes are connected to the patch and capable of electrically coupling to the patient. Sensor circuitry is coupled to the at least two electrodes and configured to measure at least two of an electrocardiogram signal of the patient, a respiration signal of the patient or an activity signal of the patient. Therapy circuitry is coupled to the at least two electrodes and configured to deliver a high-energy shock therapy for cardioversion and/or defibrillation. A processor system comprising a tangible medium and coupled to the sensor circuitry and therapy circuitry, the processor is configured to generate a treatment signal to deliver the high-energy shock therapy in response to the at least two of the electrocardiogram signal, the respiration signal or the activity signal.

In many embodiments, the adhesive patch comprises a breathable tape affixed to the at least two electrodes and the sensor circuitry and the therapy circuitry are separated from the breathable tape by a gap to allow the tape the breath.

In many embodiments, the adherent device comprises isolation circuitry to protect the sensor circuitry from the therapy circuitry when the shock therapy is delivered. The isolation circuitry may comprise at least one of a capacitor or an electrical switch.

In many embodiments, the processor system comprises a first processor comprising a tangible medium attached to the adherent patch and a second processor comprising a tangible medium at a remote center. The processor system can be configured to combine the at least two of the electrocardiogram signal, the respiration signal or the activity signal. The processor system can be configured to continuously monitor, store and transmit to a remote center the at least two of the electrocardiogram signal, the respiration signal or the activity signal in response to the treatment signal. The processor system can be configured to deliver the high-energy therapy and alert a physician in response to an adverse cardiac event. The processor system can be configured to detect at least one of a T-wave alternans, a pulsus alternans, an autonomic imbalance, a heart rate variability in response to the at least two of the electrocardiogram signal, the respiration signal or the activity signal.

In many embodiments, combining comprises the processor system using the at least two of the electrocardiogram signal, the hydration signal, the respiration signal or the activity signal to look up a value in a previously existing array. In many embodiments, combining may also comprise at least one of adding, subtracting, multiplying, scaling, or dividing the at least two of the electrocardiogram signal, the respiration signal, or the activity signal. In some embodiments at least two of the electrocardiogram signal, the accelerometer signal, or the respiration signal are combined with at least one of a weighted combination, a tiered combination or a logic gated combination, a time weighted combination or a rate of change.

In many embodiments, the processor system can be configured to loop record the at least two of the electrocardiogram signal, the respiration signal or the activity signal for diagnosis in response to the treatment signal. The processor system can be configured to acquire the electrocardiogram signal with a high sampling rate in response to the treatment signal for a period of time before the shock therapy is delivered.

In many embodiments, the processor system is configured to acquire the electrocardiogram signal with a high sampling rate for a period to time in response to the at least two of the electrocardiogram signal, the respiration signal or the activity signal. The processor system can be configured to detect an event comprising at least one of an atrial fibrillation in response to the electrocardiogram signal or an acute myocardial infarction in response to an ST segment elevation of the electrocardiogram signal.

In many embodiments, the processor system is configured to monitor the electrocardiogram signal and an alert at least one of a remote center, a physician, emergency responder, or family/caregiver when the shock therapy is delivered.

In many embodiments, the processor system is configured to determine a tiered response to the at least two of the electrocardiogram signal, the respiration signal or the activity signal. The tiered response may comprise a first tier to deliver the shock therapy comprising defibrillation, a second tier to deliver low voltage cardioversion and a third tier to deliver anti-tachycardia pacing. The processor system can be configured to measure the electrocardiogram signal after the shock therapy is delivered and escalate the therapy to another tier in response to the electrocardiogram signal.

In many embodiments, wireless communication circuitry is configured to transmit the at least two of the electrocardiogram signal, the respiration signal or the activity signal in real time in response to the treatment signal.

In many embodiments, the at least two electrodes comprise at least three electrodes and the sensor circuitry is coupled to the at least three electrodes to measure at least two vectors of the electrocardiogram signal, which can improve the specificity and the sensitivity of the delivered therapy. The at least two electrodes define a line and the least three electrodes comprise an electrode positioned away from the line to measure the at least two vectors of the electrocardiogram signal. The at least three electrodes may comprise a substantially orthogonal arrangement to measure two substantially orthogonal the at least two vectors of the electrocardiogram signal. The processor can be configured to calculate an additional vector of the electrocardiogram signal in response to the at least two vectors.

In many embodiments, the processor is configured to generate a record signal to record at least the electrocardiogram signal with high resolution for an arrhythmia log in response to the at least two of the electrocardiogram signal, the respiration signal or the activity signal. The processor can be configured to generate the record signal before the treatment in response to the at least two of the electrocardiogram signal, the respiration signal or the activity signal.

In another aspect, embodiments of the present invention provide a method of monitoring and treating a patient. An adhesive patch is adhered to a skin of the patient such that at least two electrodes connected to the patch are electrically coupled to the patient. At least two of an electrocardiogram signal of the patient, a respiration signal of the patient or an activity signal of the patient are measured with sensor circuitry coupled to the at least two electrodes. A high-energy shock therapy is delivered for cardioversion and/or defibrillation with therapy circuitry coupled to the at least two electrodes. A treatment signal is generated to deliver the high-energy shock therapy in response to the at least two of the electrocardiogram signal, the respiration signal or the activity signal, in many embodiments with a processor system comprising a tangible medium and coupled to the sensor circuitry and therapy circuitry.

In many embodiments, the adhesive patch comprises a breathable tape affixed to the at least two electrodes and the sensor circuitry and the therapy circuitry are separated from the breathable tape by a gap such that the tape the breathes when adhered to the patient. The sensor circuitry can be isolated from the electrodes and the therapy circuitry with isolation circuitry when the shock therapy is delivered.

In many embodiments, the processor system comprises a first processor comprising a tangible medium attached to the adherent patch and a second processor comprising a tangible medium at a remote center, and the first processor generates the treatment signal with instructions from the second processor. The processor system can combine the at least two of the electrocardiogram signal, the respiration signal or the activity signal.

In many embodiments, combining comprises the processor system using the at least two of the electrocardiogram signal, the hydration signal, the respiration signal or the activity signal to look up a value in a previously existing array. In many embodiments, combining may also comprise at least one of adding, subtracting, multiplying, scaling, or dividing the at least two of the electrocardiogram signal, the respiration signal, or the activity signal. In some embodiments at least two of the electrocardiogram signal, the accelerometer signal, or the respiration signal are combined with at least one of a weighted combination, a tiered combination or a logic gated combination, a time weighted combination or a rate of change.

In many embodiments, the processor system continuously monitors, stores and transmits to a remote center the at least two of the electrocardiogram signal, the respiration signal or the activity signal in response to the treatment signal.

In many embodiments, the processor system delivers the high-energy therapy and alerts a physician in response to an adverse cardiac event.

In many embodiments, the processor system detects at least one of a T-wave alternans, a pulsus alternans, an autonomic imbalance, a heart rate variability in response to the at least two of the electrocardiogram signal, the respiration signal or the activity signal.

In many embodiments, the processor system loop records the at least two of the electrocardiogram signal, the respiration signal or the activity signal for diagnosis in response to the treatment signal. The processor system can acquire the electrocardiogram signal with a high sampling rate in response to the treatment signal for a period of time before the shock therapy is delivered.

In many embodiments, the processor system can acquire the electrocardiogram signal with a high sampling rate for a period to time in response to the at least two of the electrocardiogram signal, the respiration signal or the activity signal.

In many embodiments, the processor system detects an event comprising at least one of an atrial fibrillation in response to the electrocardiogram signal or an acute myocardial infarction in response to an ST segment elevation of the electrocardiogram signal.

In many embodiments, the processor system monitors the electrocardiogram signal and alerts at least one of a remote center, a physician, emergency responder, or family/caregiver when the shock therapy is delivered.

In many embodiments, the processor system can determines a tiered response to the at least two of the electrocardiogram signal, the respiration signal or the activity signal. The tiered response can comprise a first tier to deliver the shock therapy comprising defibrillation, a second tier to deliver low voltage cardioversion and a third tier to deliver anti-tachycardia pacing. The electrocardiogram signal can be recorded after the shock therapy is delivered and processor can escalates the therapy to another tier in response to the electrocardiogram signal.

In many embodiments, wireless communication circuitry transmits the at least two of the electrocardiogram signal, the respiration signal or the activity signal in real time in response to the treatment signal.

In many embodiments, the at least two electrodes comprise at least three electrodes and the sensor circuitry is coupled to the at least three electrodes to measure at least two vectors of the electrocardiogram signal. The at least two electrodes can define a line and the least three electrodes can comprise an electrode positioned away from the line to measure the at least two vectors of the electrocardiogram signal. The at least three electrodes may comprise a substantially orthogonal arrangement to measure two substantially orthogonal vectors the electrocardiogram signal. The processor system can calculates an additional vector of the electrocardiogram signal in response to the at least two vectors.

In many embodiments, the processor system can generate a record signal in response to the at least two of the electrocardiogram signal, the respiration signal or the activity signal. The processor can record the at least two at least the electrocardiogram signal, the respiration signal of the patient or the activity signal of the patient with high resolution for an arrhythmia log in response to the record signal.

In many embodiments, the processor system can generate a record signal to record at least the electrocardiogram signal with high resolution for an arrhythmia log in response to the at least two of the electrocardiogram signal, the respiration signal or the activity signal. The processor system can generate the record signal before the treatment in response to the at least two of the electrocardiogram signal, the respiration signal or the activity signal.

### BRIEF DESCRIPTION OF THE DRAWINGS

FIG. 1A shows a patient and a monitoring and treatment system comprising an adherent device, according to embodiments of the present invention;

FIG. 1B shows a bottom view of the adherent device as in FIG. 1A comprising an adherent patch;

FIG. 1B1 shows a bottom view of adherent device comprising an adherent patch 100B1 with at least four electrodes, according to embodiments of the present invention;

FIG. 1C shows a top view of the adherent patch, as in FIG. 1B;

FIG. 1D shows a printed circuit boards and electronic components over the adherent patch, as in FIG. 1C;

FIG. 1D1 shows an equivalent circuit that can be used to determine optimal frequencies for determining patient hydration, according to embodiments of the present invention;

FIG. 1E shows batteries positioned over the printed circuit board and electronic components as in FIG. 1D;

FIG. 1F shows a top view of an electronics housing and a breathable cover over the batteries, electronic components and printed circuit board as in FIG. 1E;

FIG. 1G shows a side view of the adherent device as in FIGS. 1A to 1F;

FIG. 1H shown a bottom isometric view of the adherent device as in FIGS. 1A to 1G;

FIGS. 1I and 1J show a side cross-sectional view and an exploded view, respectively, of the adherent device as in FIGS. 1A to 1H;

FIG. 1K shows at least one electrode configured to electrically couple to a skin of the patient through a breathable tape, according to embodiments of the present invention;

FIG. 2A shows an adherent measurement and treatment device comprising an adherent patch with at least three electrodes to measure at least two vectors of the electrocardiogram signal, according to embodiments of the present invention;

FIG. 2B shows an adherent measurement and treatment device comprising an adherent patch with at least four electrodes to measure at least two vectors of the electrocardiogram signal; and

FIG. 3A shows a method of monitoring and treating a patient with an adherent device, according to embodiments of the present invention.

### DETAILED DESCRIPTION OF THE INVENTION

Embodiments of the present invention relate to patient monitoring. Although embodiments make specific reference to monitoring and treatment with an adherent patch, the system, methods and devices described herein may be applicable to any application in which physiological monitoring and treatment are used, for example wireless physiological monitoring and treatment for extended periods.

Embodiments of the present invention provide an external, adherent device, which can be affixed to the patient's thorax, and comprises multiple physiological sensors. The device can wirelessly communicate with a remote center, either directly or indirectly via an intermediate device. The system can continuously monitor physiologic variables and issue patient and/or physician alerts when appropriate, and may provide therapy with high energy electrical shocks if appropriate.

In many embodiments, the adherent devices described herein may be used for 90 day monitoring, or more, and may comprise completely disposable components and/or reusable components, and can provide reliable data acquisition and transfer. In many embodiments, the patch is configured for patient comfort, such that the patch can be worn and/or tolerated by the patient for extended periods, for example 90 days or more. In many embodiments, the adherent patch comprises a tape, which comprises a material, preferably breathable, with an adhesive, such that trauma to the patient skin can be minimized while the patch is worn for the extended period. In many embodiments, the printed circuit board comprises a flex printed circuit board that can flex with the patient to provide improved patient comfort.

The device would contain a full suite of sensors for tachyarrhythmia detection, primarily relying on the electrocardiogram to detect the incidence of a life-threatening arrhythmia, such as VT/VF. It would contain the capability for delivering a small number of high-energy shocks for external cardioversion/defibrillation. It may also contain the capability for external cardiac pacing. The device would detect arrhythmia initiation and respond by providing tiered CRM therapy: anti-tachycardia pacing, low-voltage cardioversion, and/or high-voltage defibrillation. Following therapy delivery, the arrhythmia would be redetected and the therapy would be escalated as appropriate.

Therapy delivery may also be in the form of transcutaneous neural stimulation, which may be delivered alone or in conjunction with previously listed CRM therapies.

In many embodiments, a patient with electrophysiological disorders can be monitored safely as the device can provide intervention if needed. Patients with a transient physiology, such as following a myocardial infarction, may wear the device as a bridge to implantation of a cardiac rhythm management device, such as an implantable cardio defibrillator (ICD). In many embodiments, the adherent device may comprise a temporary, “safety net” solution for patients at risk for SCD. In some embodiments, the device may allow for outpatient recovery following a myocardial infarction (MI). The device can be used to assess the need for a permanent implant and can be beneficial in patients who may require intervention while such determination is made.

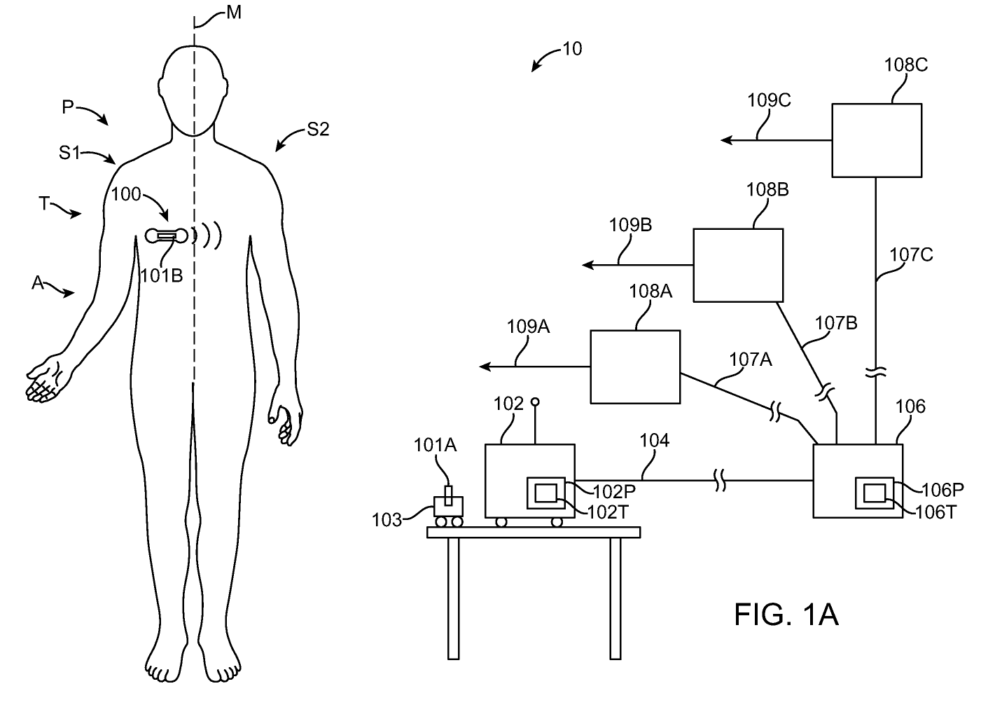

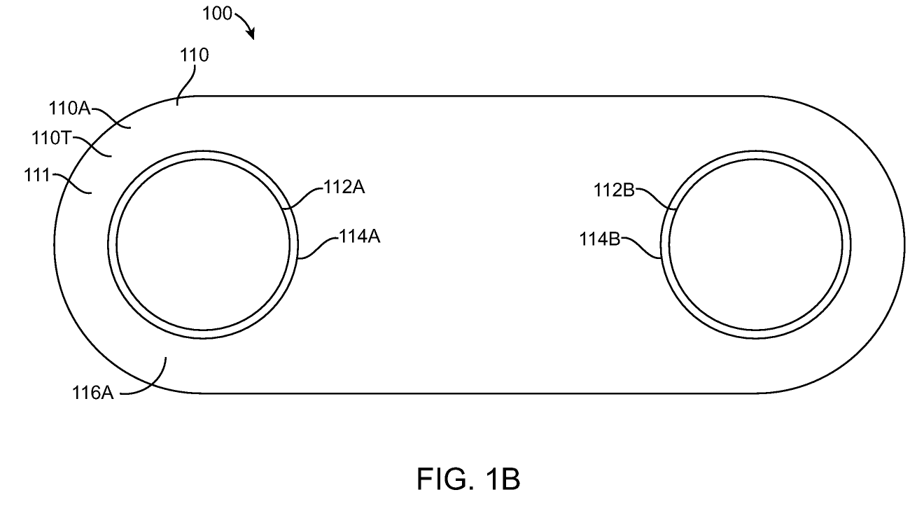

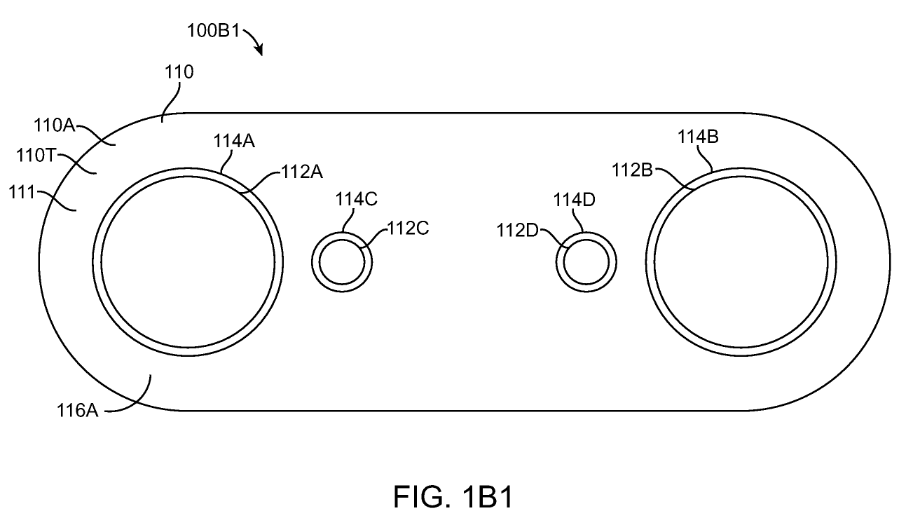

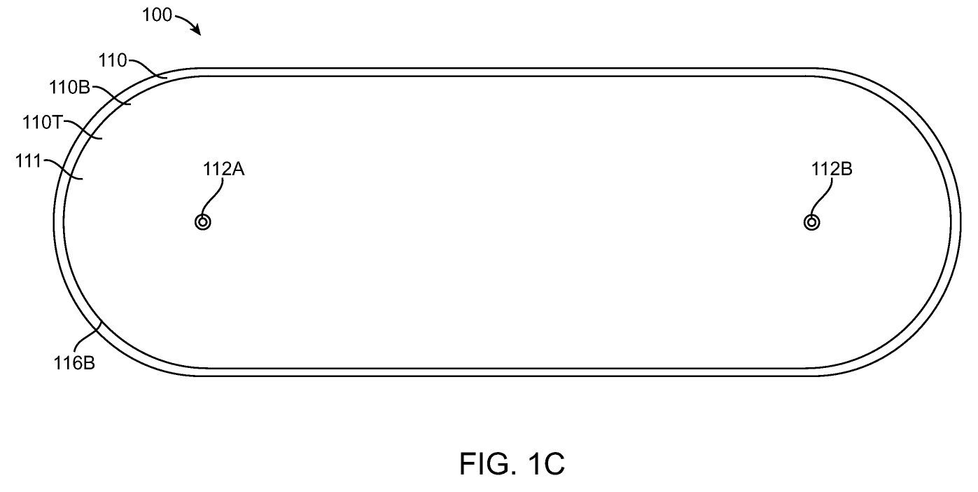

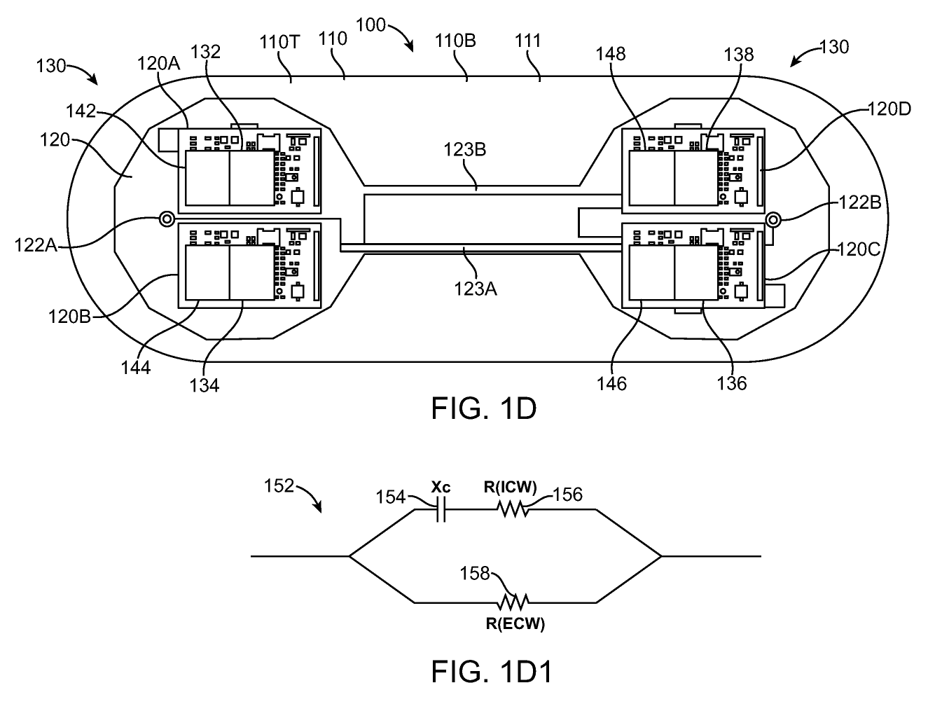

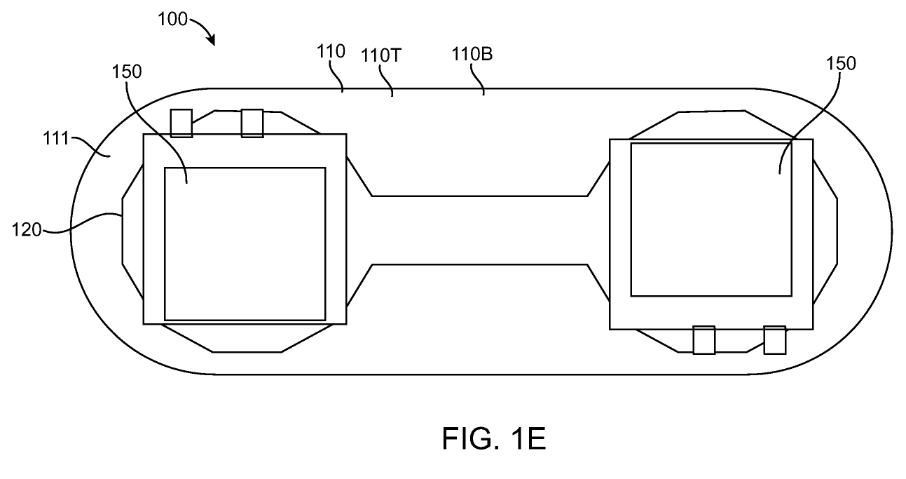

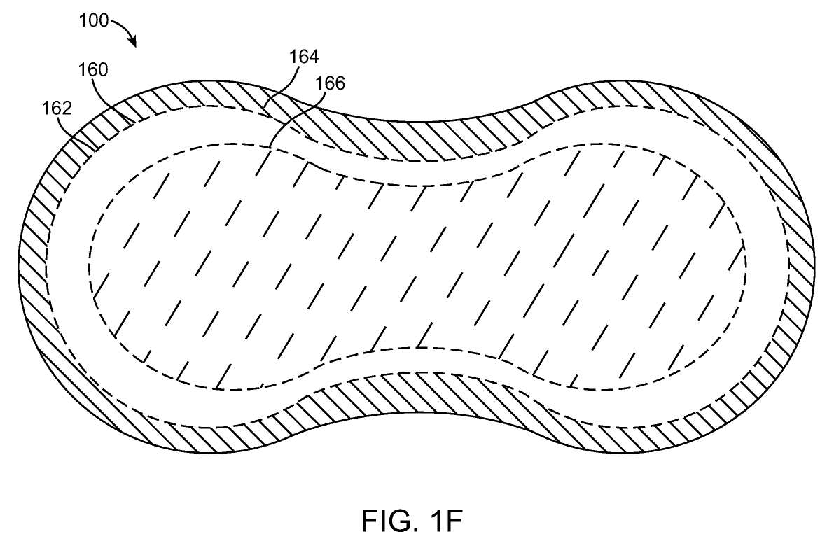

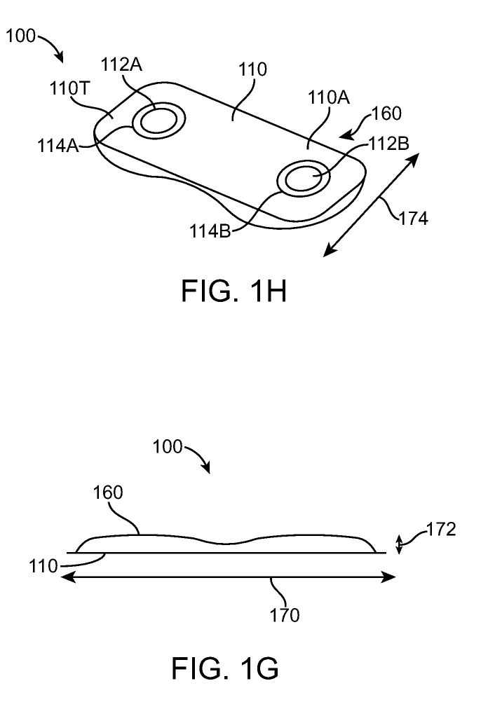

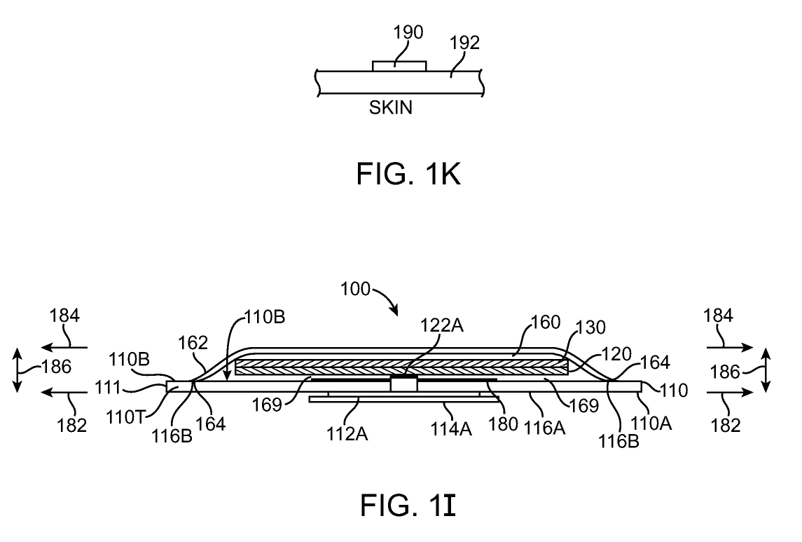

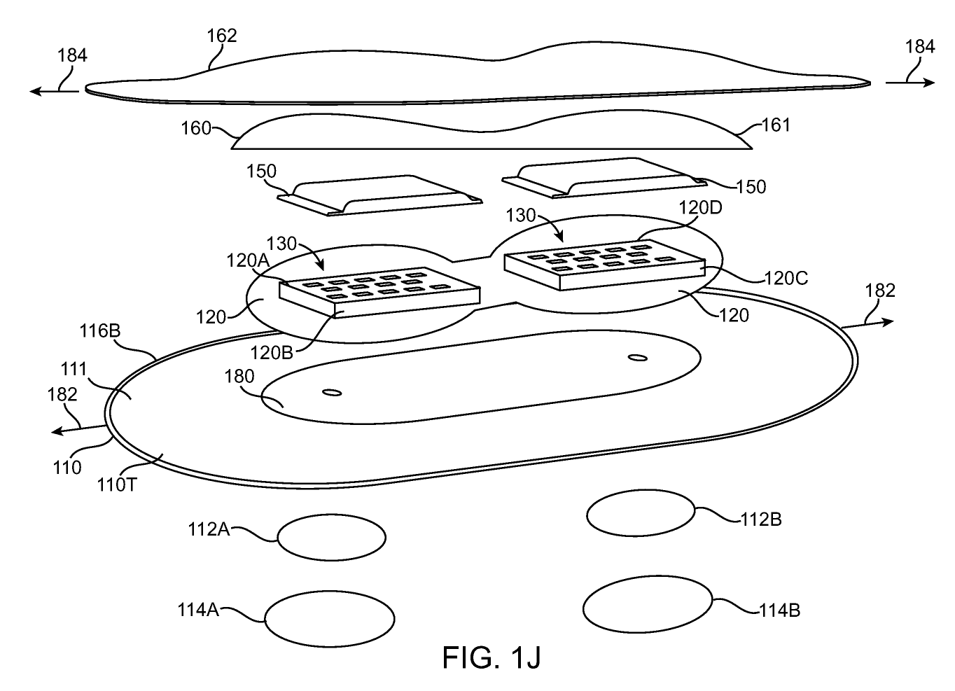

FIG. 1A shows a patient P and a monitoring and treatment system 10. Patient P comprises a midline M, a first side S1, for example a right side, and a second side S2, for example a left side. Monitoring and treatment system 10 comprises an adherent device 100. Adherent device 100 can be adhered to a patient P at many locations, for example thorax T of patient P. In many embodiments, the adherent device may adhere to one side of the patient, from which side data can be collected. Work in relation with embodiments of the present invention suggests that location on a side of the patient can provide comfort for the patient while the device is adhered to the patient.

Monitoring and treatment system 10 includes components to transmit data to a remote center 106. Remote center 106 can be located in a different building from the patient, for example in the same town as the patient, and can be located as far from the patient as a separate continent from the patient, for example the patient located on a first continent and the remote center located on a second continent. Adherent device 100 can communicate wirelessly to an intermediate device 102, for example with a single wireless hop from the adherent device on the patient to the intermediate device. Intermediate device 102 can communicate with remote center 106 in many ways, for example with an Internet connection and/or with a cellular connection. The remote center can be located in many places, for example in the same country or a different country as the patient and/or in the same continent and/or a different continent than the patient. In many embodiments, monitoring system 10 comprises a distributed processing system. The distributed processing system may comprise at least one processor on device 100, at least one processor 102P on intermediate device 102, and at least one processor 106P at remote center 106, each of which processors is in electronic communication with the other processors. At least one processor 102P comprises a tangible medium 102T, and at least one processor 106P comprises a tangible medium 106T. Remote processor 106P may comprise a backend server located at the remote center. Remote center 106 can be in communication with a health care provider 108A with a communication system 107A, such as the Internet, an intranet, phone lines, wireless and/or satellite phone. Health care provider 108A, for example a family member, can be in communication with patient P with a communication, for example with a two way communication system, as indicated by arrow 109A, for example by cell phone, email, landline. Remote center 106 can be in communication with a health care professional, for example a physician 108B, with a communication system 107B, such as the Internet, an intranet, phone lines, wireless and/or satellite phone. Physician 108B can be in communication with patient P with a communication, for example with a two way communication system, as indicated by arrow 109B, for example by cell phone, email, landline. Remote center 106 can be in communication with an emergency responder 108C, for example a 911 operator and/or paramedic, with a communication system 107C, such as the Internet, an intranet, phone lines, wireless and/or satellite phone. Emergency responder 108C can travel to the patient as indicated by arrow 109C. Thus, in many embodiments, monitoring and treatment system 10 comprises a closed loop system in which patient care can be monitored and implemented from the remote center in response to signals from the adherent device.

In many embodiments, the adherent device may continuously monitor physiological parameters, communicate wirelessly with a remote center, and provide alerts when necessary. The system may comprise an adherent patch, which attaches to the patient's thorax and contains sensing electrodes, battery, memory, logic, and wireless communication capabilities. In some embodiments, the patch can communicate with the remote center, via the intermediate device in the patient's home. Remote center 106 can receive the patient data and applies the prediction algorithm. When a flag is raised, the center may communicate with the patient, hospital, nurse, and/or physician to allow for therapeutic intervention to prevent decompensation.

The adherent device may be affixed and/or adhered to the body in many ways. For example, with at least one of the following an adhesive tape, a constant-force spring, suspenders around shoulders, a screw-in microneedle electrode, a pre-shaped electronics module to shape fabric to a thorax, a pinch onto roll of skin, or transcutaneous anchoring. Patch and/or device replacement may occur with a keyed patch (e.g. two-part patch), an outline or anatomical mark, a low-adhesive guide (place guide|remove old patch|place new patch|remove guide), or a keyed attachment for chatter reduction. The patch and/or device may comprise an adhesiveless embodiment (e.g. chest strap), and/or a low-irritation adhesive model for sensitive skin. The adherent patch and/or device can comprise many shapes, for example at least one of a dogbone, an hourglass, an oblong, a circular or an oval shape.

In many embodiments, the adherent device may comprise a reusable electronics module with replaceable patches (the module collects cumulative data for approximately 90 days) and/or the entire adherent component (electronics+patch) may be disposable. In a completely disposable embodiment, a “baton” mechanism may be used for data transfer and retention, for example baton transfer may include baseline information. In some embodiments, the device may have a rechargeable module, and may use dual battery and/or electronics modules, wherein one module 101A can be recharged using a charging station 103 while the other module 101B is placed on the adherent patch with connectors. In some embodiments, the intermediate device 102 may comprise the charging module, data transfer, storage and/or transmission, such that one of the electronics modules can be placed in the intermediate device for charging and/or data transfer while the other electronics module is worn by the patient.

System 10 can perform the following functions: initiation, programming, measuring, storing, analyzing, communicating, predicting, and displaying. The adherent device may contain a subset of the following physiological sensors: bioimpedance, respiration, respiration rate variability, heart rate (ave, min, max), heart rhythm, HRV, HRT, heart sounds (e.g. S3), respiratory sounds, blood pressure, activity, posture, wake/sleep, orthopnea, temperature/heat flux, and weight. The activity sensor may comprise one or more of the following: ball switch, accelerometer, minute ventilation, HR, bioimpedance noise, skin temperature/heat flux, BP, muscle noise, posture.

The adherent device can wirelessly communicate with remote center 106. In some embodiments, the communication may occur directly (via a cellular or Wi-Fi network), or indirectly through intermediate device 102. Intermediate device 102 may comprise multiple devices which can communicate wired or wirelessly to relay data to remote center 106.

In many embodiments, instructions are transmitted from remote site 106 to a processor supported with the adherent patch on the patient, and the processor supported with the patient can receive updated instructions for the patient treatment and/or monitoring, for example while worn by the patient.

FIG. 1B shows a bottom view of adherent device 100 as in FIG. 1A comprising an adherent patch 110. Adherent patch 110 comprises a first side, or a lower side 110A, that is oriented toward the skin of the patient when placed on the patient. In many embodiments, adherent patch 110 comprises a tape 110T which is a material, preferably breathable, with an adhesive 116A. In many embodiments, tape 110T comprises a backing 111. Patient side 110A comprises adhesive 116A to adhere the patch 110 and adherent device 100 to patient P. Electrodes 112A and 112B may be affixed to adherent patch 110. In many embodiments, at least two electrodes are attached to the patch. In some embodiments, the patch comprises two electrodes, for example two electrodes to measure the electrocardiogram (ECG) of the patient. Gel 114A and gel 114B can each be positioned over electrodes 112A and 112B, respectively, to provide electrical conductivity between the electrodes and the skin of the patient. In many embodiments, the electrodes can be affixed to the patch 110, for example with known methods and structures such as rivets, adhesive, stitches, etc. In many embodiments, patch 110 comprises a breathable material to permit air and/or vapor to flow to and from the surface of the skin.

FIG. 1B1 shows a bottom view of adherent device comprising an adherent patch 100B1 with at least four electrodes. Patch 100B1 can be used to measure patient impedance, for example four pole impedance, and used for cardiac rhythm management. Patch 100B1 includes structures similar to adherent patch 100. In addition to electrode 112A and electrode 112B, adherent patch 100B1 comprises electrode 112C and electrode 112D. The electrodes can be arranged such that electrode 112A and electrode 112B comprise outer electrodes and electrode 112C and electrode 112D comprise inner electrodes. Gel 114C can be disposed over electrode 112C, and gel 114D can be disposed over electrode 112D.

FIG. 1C shows a top view of the adherent patch 100, as in FIG. 1B. Adherent patch 100 comprises a second side, or upper side 110B. In many embodiments, electrodes 110A and 110B extend from lower side 110A through the adherent patch to upper side 110B. An adhesive 116B can be applied to upper side 110B to adhere structures, for example a breathable cover, to the patch such that the patch can support the electronics and other structures when the patch is adhered to the patient. The PCB can comprise completely flex PCB, rigid PCB combined flex PCB and/or rigid PCB boards connected by cable.

FIG. 1D shows a printed circuit boards and electronic components over adherent patch 110, as in FIG. 1C. In some embodiments, a printed circuit board (PCB), for example flex PCB 120, may be connected to upper side 100B of patch 110 with connectors 122A and 122B. Flex PCB 120 can include traces 123A and 123B that extend to connectors 122A and 122B, respectively, on the flex PCB. Connectors 122A and 122B can be positioned on flex PCB 120 in alignment with electrodes 112A and 112B so as to electrically couple the flex PCB with the electrodes. In some embodiments, connectors 122A and 122B may comprise insulated wires and/or a film with conductive ink that provide strain relief between the PCB and the electrodes. In some embodiments, additional PCB's, for example rigid PCB's 120A, 120B, 120C and 120D, can be connected to flex PCB 120. Electronic components 130 can be connected to flex PCB 120 and/or mounted thereon. In some embodiments, electronic components 130 can be mounted on the additional PCB's.

Electronic components 130 comprise components for therapy and to take physiologic measurements, transmit data to remote center 106 and receive commands from remote center 106. In many embodiments, electronics components 130 may comprise known low power circuitry, for example complementary metal oxide semiconductor (CMOS) circuitry components. Electronics components 130 comprise an activity sensor and activity circuitry 134, impedance circuitry 136 and electrocardiogram circuitry, for example ECG circuitry 136. In some embodiments, electronics circuitry 130 may comprise a microphone and microphone circuitry 142 to detect an audio signal from within the patient, and the audio signal may comprise a heart sound and/or a respiratory sound, for example an S3 heart sound and a respiratory sound with rales and/or crackles.

Electronics circuitry 130 may comprise a temperature sensor, for example a thermistor in contact with the skin of the patient, and temperature sensor circuitry 144 to measure a temperature of the patient, for example a temperature of the skin of the patient.

Work in relation to embodiments of the present invention suggests that skin temperature may effect impedance and/or hydration measurements, and that skin temperature measurements may be used to correct impedance and/or hydration measurements. In some embodiments, increase in skin temperature or heat flux can be associated with increased vaso-dilation near the skin surface, such that measured impedance measurement decreased, even through the hydration of the patient in deeper tissues under the skin remains substantially unchanged. Thus, use of the temperature sensor can allow for correction of the hydration signals to more accurately assess the hydration, for example extra cellular hydration, of deeper tissues of the patient, for example deeper tissues in the thorax.

Electronics circuitry 130 may comprise a processor 146. Processor 146 comprises a tangible medium, for example read only memory (ROM), electrically erasable programmable read only memory (EEPROM) and/or random access memory (RAM). Processor 146 may comprise real time clock and frequency generator circuitry, for example the PIC series or microprocessors available from Microchip of Chandler, Ariz. In some embodiments, processor 136 may comprise the frequency generator and real time clock. The processor can be configured to control a collection and transmission of data from the impedance circuitry electrocardiogram circuitry and the accelerometer. In many embodiments, device 100 comprise a distributed processor system, for example with multiple processors on device 100.

Electronics circuitry 130 comprises high energy shock circuitry 148 to deliver a sequence of high energy shocks to the patient. High energy shock circuitry may comprise known circuits, for example voltage converters, to deliver the high energy shocks to the electrodes. High energy shock circuitry 148 may comprise isolation circuitry to isolate and/or decouple the measurement circuitry, described above, from the electrodes when the high energy shocks are delivered. In some embodiments, the measurement circuitry, for example impedance and electrocardiogram circuitry, may comprise the isolation circuitry. In many embodiments, the isolation circuitry may comprises known electronics to isolate a circuit, for example switches, and may comprise capacitors.

In many embodiments, electronics components 130 comprise wireless communications circuitry 132 to communicate with remote center 106. The wireless communication circuitry can be coupled to the impedance circuitry, the electrocardiogram circuitry and the accelerometer to transmit to a remote center with a communication protocol at least one of the hydration signal, the electrocardiogram signal or the inclination signal. In specific embodiments, wireless communication circuitry is configured to transmit the hydration signal, the electrocardiogram signal and the inclination signal to the remote center with a single wireless hop, for example from wireless communication circuitry 132 to intermediate device 102. The communication protocol comprises at least one of Bluetooth, Zigbee, WiFi, WiMax, IR, amplitude modulation or frequency modulation. In many embodiments, the communications protocol comprises a two way protocol such that the remote center is capable of issuing commands to control data collection.

Intermediate device 102 may comprise a data collection system to collect and store data from the wireless transmitter. The data collection system can be configured to communicate periodically with the remote center. The data collection system can transmit data in response to commands from remote center 106 and/or in response to commands from the adherent device.

Activity sensor and activity circuitry 134 can comprise many known activity sensors and circuitry. In many embodiments, the accelerometer comprises at least one of a piezoelectric accelerometer, capacitive accelerometer or electromechanical accelerometer. The accelerometer may comprises a 3-axis accelerometer to measure at least one of an inclination, a position, an orientation or acceleration of the patient in three dimensions. Work in relation to embodiments of the present invention suggests that three dimensional orientation of the patient and associated positions, for example sitting, standing, lying down, can be very useful when combined with data from other sensors, for example ECG data and/or hydration data.

Impedance circuitry 136 can generate both hydration data and respiration data. In many embodiments, impedance circuitry 136 is electrically connected to electrodes 112A, 112B, 112C and 112D such that electrodes 112A and 112B comprise outer electrodes that are driven with a current, or force electrodes. The current delivered between electrodes 112A and 112B generates a measurable voltage between electrodes 112C and 112D, such that electrodes 112C and 112D comprise inner electrodes, or sense electrodes that measure the voltage in response to the current from the force electrodes. The voltage measured by the sense electrodes can be used to determine the hydration of the patient.

FIG. 1D-1 shows an equivalent circuit 152 that can be used to determine optimal frequencies for measuring patient hydration. Work in relation to embodiments of the present invention indicates that the frequency of the current and/or voltage at the force electrodes can be selected so as to provide impedance signals related to the extracellular and/or intracellular hydration of the patient tissue. Equivalent circuit 152 comprises an intracellular resistance 156, or R(ICW) in series with a capacitor 154, and an extracellular resistance 158, or R(ECW). Extracellular resistance 158 is in parallel with intracellular resistance 156 and capacitor 154 related to capacitance of cell membranes. In many embodiments, impedances can be measured and provide useful information over a wide range of frequencies, for example from about 0.5 kHz to about 200 KHz. Work in relation to embodiments of the present invention suggests that extracellular resistance 158 can be significantly related extracellular fluid and to cardiac decompensation, and that extracellular resistance 158 and extracellular fluid can be effectively measured with frequencies in a range from about 0.5 kHz to about 20 kHz, for example from about 1 kHz to about 10 kHz. In some embodiments, a single frequency can be used to determine the extracellular resistance and/or fluid. As sample frequencies increase from about 10 kHz to about 20 kHz, capacitance related to cell membranes decrease the impedance, such that the intracellular fluid contributes to the impedance and/or hydration measurements. Thus, many embodiments of the present invention measure hydration with frequencies from about 0.5 kHz to about 20 kHz to determine patient hydration.

In many embodiments, impedance circuitry 136 can be configured to determine respiration of the patient. In specific embodiments, the impedance circuitry can measure the hydration at 25 Hz intervals, for example at 25 Hz intervals using impedance measurements with a frequency from about 0.5 kHz to about 20 kHz.

ECG circuitry 138 can generate electrocardiogram signals and data from electrodes 112A and 112B. In some embodiments, ECG circuitry 138 is connected to inner electrodes 112C and 122D, which may comprise sense electrodes of the impedance circuitry as described above. In some embodiments, the inner electrodes may be positioned near the outer electrodes to increase the voltage of the ECG signal measured by ECG circuitry 138. In some embodiments, the ECG circuitry can share components with the impedance circuitry.

FIG. 1E shows batteries 150 positioned over the flex printed circuit board and electronic components as in FIG. 1D. Batteries 150 may comprise rechargeable batteries that can be removed and/or recharged. In some embodiments, batteries 150 can be removed from the adherent patch and recharged and/or replaced.

FIG. 1F shows a top view of a cover 162 over the batteries, electronic components and flex printed circuit board as in FIGS. 1A to 1E. In many embodiments, an electronics housing 160 may be disposed under cover 162 to protect the electronic components, and in some embodiments electronics housing 160 may comprise an encapsulant over the electronic components and PCB. In some embodiments, cover 162 can be adhered to adherent patch 110 with an adhesive 164 on an underside of cover 162. In many embodiments, electronics housing 160 may comprise a water proof material, for example a sealant adhesive such as epoxy or silicone coated over the electronics components and/or PCB. In some embodiments, electronics housing 160 may comprise metal and/or plastic. Metal or plastic may be potted with a material such as epoxy or silicone.

Cover 162 may comprise many known biocompatible cover, casing and/or housing materials, such as elastomers, for example silicone. The elastomer may be fenestrated to improve breathability. In some embodiments, cover 162 may comprise many known breathable materials, for example polyester, polyamide, and/or elastane (Spandex). The breathable fabric may be coated to make it water resistant, waterproof, and/or to aid in wicking moisture away from the patch.

FIG. 1G shows a side view of adherent device 100 as in FIGS. 1A to 1F. Adherent device 100 comprises a maximum dimension, for example a length 170 from about 4 to 10 inches (from about 100 mm to about 250 mm), for example from about 6 to 8 inches (from about 150 mm to about 200 mm). In some embodiments, length 170 may be no more than about 6 inches (no more than about 150 mm). Adherent device 100 comprises a thickness 172. Thickness 172 may comprise a maximum thickness along a profile of the device. Thickness 172 can be from about 0.2 inches to about 0.4 inches (from about 5 mm to about 10 mm), for example about 0.3 inches (about 7.5 mm).

FIG. 1H shown a bottom isometric view of adherent device 100 as in FIGS. 1A to 1G. Adherent device 100 comprises a width 174, for example a maximum width along a width profile of adherent device 100. Width 174 can be from about 2 to about 4 inches (from about 50 mm to 100 mm), for example about 3 inches (about 75 mm).

FIGS. 1I and 1J show a side cross-sectional view and an exploded view, respectively, of adherent device 100 as in FIGS. 1A to 1H. Device 100 comprises several layers. Gel 114A, or gel layer, is positioned on electrode 112A to provide electrical conductivity between the electrode and the skin. Electrode 112A may comprise an electrode layer. Adhesive patch 110 may comprise a layer of breathable tape 110T, for example a known breathable tape, such as tricot-knit polyester fabric. An adhesive 116A, for example a layer of acrylate pressure sensitive adhesive, can be disposed on underside 110A of adherent patch 110. A gel cover 180, or gel cover layer, for example a polyurethane non-woven tape, can be positioned over patch 110 comprising the breathable tape. A PCB layer, for example flex PCB 120, or flex PCB layer, can be positioned over gel cover 180 with electronic components 130 connected and/or mounted to flex PCB 120, for example mounted on flex PCB so as to comprise an electronics layer disposed on the flex PCB layer. In many embodiments, the adherent device may comprise a segmented inner component, for example the PCB may be segmented to provide at least some flexibility. In many embodiments, the electronics layer may be encapsulated in electronics housing 160 which may comprise a waterproof material, for example silicone or epoxy. In many embodiments, the electrodes are connected to the PCB with a flex connection, for example trace 123A of flex PCB 120, so as to provide strain relive between the electrodes 112A and 112B and the PCB. Gel cover 180 can inhibit flow of gel 114A and liquid. In many embodiments, gel cover 180 can inhibit gel 114A from seeping through breathable tape 110T to maintain gel integrity over time. Gel cover 180 can also keep external moisture, for example liquid water, from penetrating through the gel cover into gel 114A while allowing moisture vapor from the gel, for example moisture vapor from the skin, to transmit through the gel cover. In many embodiments, cover 162 can encase the flex PCB and/or electronics and can be adhered to at least one of the electronics, the flex PCB or adherent patch 110, so as to protect at least the electronic components and the PCB. Cover 162 can attach to adhesive patch 110 with adhesive 1116B. Cover 162 can comprise many known biocompatible cover materials, for example silicone. Cover 162 can comprise an outer polymer cover to provide smooth contour without limiting flexibility. In many embodiments, cover 162 may comprise a breathable fabric. Cover 162 may comprise many known breathable fabrics, for example breathable fabrics as described above. In some embodiments, the breathable cover may comprise a breathable water resistant cover. In some embodiments, the breathable fabric may comprise polyester, nylon, polyamide, and/or elastane (Spandex) to allow the breathable fabric to stretch with body movement. In some embodiments, the breathable tape may contain and elute a pharmaceutical agent, such as an antibiotic, anti-inflammatory or antifungal agent, when the adherent device is placed on the patient.

The breathable cover 162 and adherent patch 110 comprising breathable tape can be configured to couple continuously for at least one week the at least one electrode to the skin so as to measure breathing of the patient. The breathable tape may comprise the stretchable breathable material with the adhesive and the breathable cover may comprises a stretchable water resistant material connected to the breathable tape, as described above, such that both the adherent patch and cover can stretch with the skin of the patient. Arrows 182 show stretching of adherent patch 110, and the stretching of adherent patch can be at least two dimensional along the surface of the skin of the patient. As noted above, connectors 122A, 122B, 122C and 122D between PCB 130 and electrodes 112A, 112B, 112C and 112D may comprise insulated wires that provide strain relief between the PCB and the electrodes, such that the electrodes can move with the adherent patch as the adherent patch comprising breathable tape stretches. Arrows 184 show stretching of cover 162, and the stretching of the cover can be at least two dimensional along the surface of the skin of the patient. Cover 162 can be attached to adherent patch 110 with adhesive 116B such that cover 162 stretches and/or retracts when adherent patch 110 stretches and/or retracts with the skin of the patient. Electronics housing 160 can be smooth and allow breathable cover 162 to slide over electronics housing 160, such that motion and/or stretching of cover 162 is slidably coupled with housing 160. The printed circuit board can be slidably coupled with adherent patch 110 that comprises breathable tape 110T, such that the breathable tape can stretch with the skin of the patient when the breathable tape is adhered to the skin of the patient. Electronics components 130 can be affixed to printed circuit board 120, for example with solder, and the electronics housing can be affixed over the PCB and electronics components, for example with dip coating, such that electronics components 130, printed circuit board 120 and electronics housing 160 are coupled together. Electronics components 130, printed circuit board 120, and electronics housing 160 are disposed between the stretchable breathable material of adherent patch 110 and the stretchable water resistant material of cover 160 so as to allow the adherent patch 110 and cover 160 to stretch together while electronics components 130, printed circuit board 120, and electronics housing 160 do not stretch substantially, if at all. This decoupling of electronics housing 160, printed circuit board 120 and electronic components 130 can allow the adherent patch 110 comprising breathable tape to move with the skin of the patient, such that the adherent patch can remain adhered to the skin for an extended time of at least one week, for example two or more weeks.

The breathable tape of adhesive patch 110 may comprise a first mesh with a first porosity and gel cover 180 may comprise a breathable tape with a second porosity, in which the second porosity is less than the first porosity to minimize, and even inhibit, flow of the gel through the breathable tape. The gel cover may comprise a polyurethane film with the second porosity.

An air gap 169 may extend from adherent patch 110 to the electronics module and/or PCB, so as to provide patient comfort. Air gap 169 allows adherent patch 110 and breathable tape 110T to remain supple and move, for example bend, with the skin of the patient with minimal flexing and/or bending of printed circuit board 120 and electronic components 130, as indicated by arrows 186. Printed circuit board 120 and electronics components 130 that are separated from the breathable tape 110T with air gap 169 can allow the skin to release moisture as water vapor through the breathable tape, gel cover, and breathable cover. This release of moisture from the skin through the air gap can minimize, and even avoid, excess moisture, for example when the patient sweats and/or showers.

In many embodiments, the adherent device comprises a patch component and at least one electronics module. The patch component may comprise adhesive patch 110 comprising the breathable tape with adhesive coating 116A, at least one electrode, for example electrode 112A and gel 114A, for example a gel coating. The at least one electronics module can be separable from the patch component. In many embodiments, the at least one electronics module comprises the flex printed circuit board 120, electronic components 130, electronics housing 160 and cover 162, such that the flex printed circuit board, electronic components, electronics housing and cover are reusable and/or removable for recharging and data transfer, for example as described above. In many embodiments, adhesive 116B is coated on upper side 110A of adhesive patch 110B, such that the electronics module can be adhered to and/or separated from the adhesive component. In specific embodiments, the electronic module can be adhered to the patch component with a releasable connection, for example with Velcro™, a known hook and loop connection, and/or snap directly to the electrodes. Two electronics modules can be provided, such that one electronics module can be worn by the patient while the other is charged, as described above. Monitoring with multiple adherent patches for an extended period is described in U.S. Pat. App. No. 60/972,537, the full disclosure of which has been previously incorporated herein by reference. Many patch components can be provided for monitoring and rhythm therapy over the extended period. For example, about 12 patches can be used to monitor and provide therapy for the patient for at least 90 days with at least one electronics module, for example with two reusable electronics modules.

In many embodiments, at least one electrode 112A can extend through at least one aperture 180A in the breathable tape 110 and gel cover 180.

In many embodiments, the adherent device comprises a patch component and at least one electronics module. The patch component may comprise adhesive patch 110 comprising the breathable tape with adhesive coating 116A, at least one electrode 114A and gel 114, for example a gel coating. The at least one electronics module can be is separable from the patch component. In many embodiments, the at least one electronics module comprises the flex printed circuit board 120, electronic component 130, electronics housing 160 and waterproof cover 162, such that the flex printed circuit board, electronic components electronics housing and water proof cover are reusable and/or removable for recharging and data transfer, for example as described above. In many embodiments, adhesive 116B is coated on upper side 110A of adhesive patch 110B, such that the electronics module, or electronics layers, can be adhered to and/or separated from the adhesive component, or adhesive layers. In specific embodiments, the electronic module can be adhered to the patch component with a releasable connection, for example with Velcro™, a known hook and loop connection, and/or snap directly to the electrodes. In some embodiments, two electronics modules can be provided, such that one electronics module can be worn by the patient while the other is charged as described above.

In some embodiments, the adhesive patch may comprise a medicated patch that releases a medicament, such as antibiotic, beta-blocker, ACE inhibitor, diuretic, or steroid to reduce skin irritation. In some embodiments, the adhesive patch may comprise a thin, flexible, breathable patch with a polymer grid for stiffening. This grid may be anisotropic, may use electronic components to act as a stiffener, may use electronics-enhanced adhesive elution, and may use an alternating elution of adhesive and steroid.

FIG. 1K shows at least one electrode 190 configured to electrically couple to a skin of the patient through a breathable tape 192. In many embodiments, at least one electrode 190 and breathable tape 192 comprise electrodes and materials similar to those described above. Electrode 190 and breathable tape 192 can be incorporated into adherent devices as described above, so as to provide electrical coupling between the skin an electrode through the breathable tape, for example with the gel.

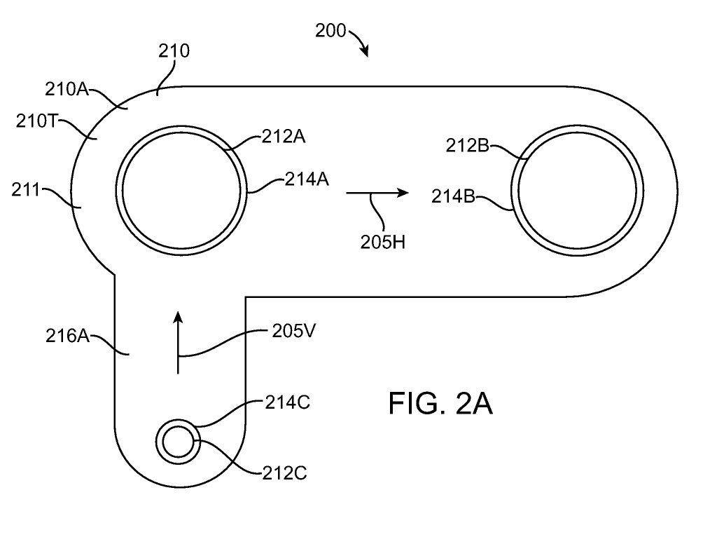

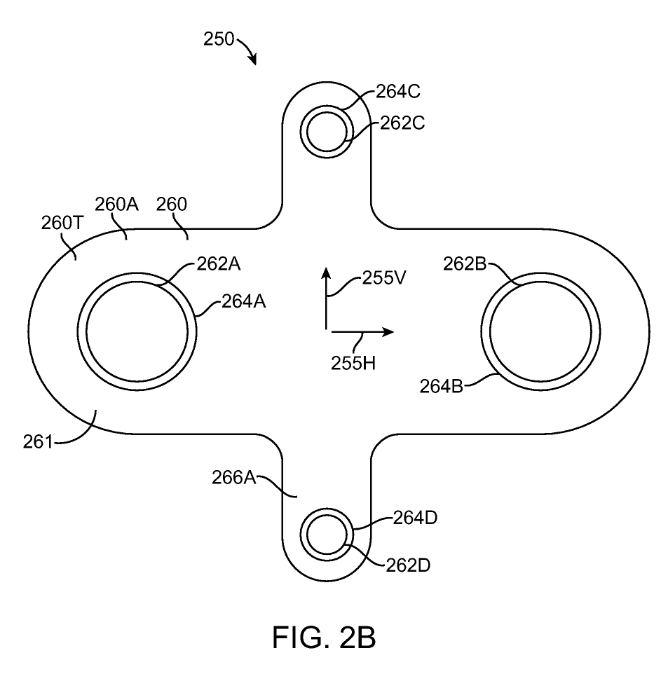

FIG. 2A shows an adherent patch 210 with at least three electrodes to measure at least two vectors of the electrocardiogram signal. Adherent patch 210 can be incorporated into the monitoring and therapy device as described above. Device 200 may comprise the circuitry, casing, housing, electrodes and structures described above. Adherent patch 210 may comprise a breathable tape 210T with a lower side 210A, or patient side, oriented toward the skin of the patient. Lower side 210A may comprise an adhesive 216A. Breathable tape 210T may comprise a backing 211. A first electrode 212A and a second electrode 212B can measure an electrocardiogram signal and treat the patient with a high voltage shock therapy as described above. Adherent patch 210 comprises a third electrode 212C that may comprise a measurement electrode. Gel 214A, gel 214B and gel 214C can each be positioned over electrodes 112A, 112B and 112C, respectively. Sensor circuitry as described above can be coupled to the at least three electrodes to measure at least two vectors of the electrocardiogram signal.

A first vector may comprise a horizontal vector 205H that corresponds to a measurement axis extending from electrode 262A to electrode 262B. A second vector may comprise a vertical vector 205V that corresponds measurement axis extending from electrode 262C to electrode 262D. In many embodiments, the vectors of the electrocardiogram signal can be calculated with the processor on the adherent device, for example with known methods of calculating vectors. The first electrode 212A and second electrode 212B can define a line, and the third electrode 212C comprises an electrode positioned away from the line to measure the at least two vectors of the electrocardiogram signal. In specific embodiments, the at least three electrodes comprise a substantially orthogonal arrangement to measure two substantially orthogonal vectors of the electrocardiogram signal. The processor system can be configured to calculate an additional vector of the electrocardiogram signal in response to the at least two vectors, for example with the processor on the adherent device. First electrode 212A and second electrode 212B can be larger than third electrode 212C, for example at least about twice the diameter and can at least three to four times the diameter.

FIG. 2B shows an adherent measurement and treatment device comprising an adherent patch with at least four electrodes to measure at least two vectors of the electrocardiogram signal. Adherent patch 260 can be incorporated into the monitoring and therapy device as described above. Device 250 may comprise the circuitry, casing, housing, electrodes and structures described above. Adherent patch 260 may comprise a breathable tape 260T with a lower side 260A, or patient side, oriented toward the skin of the patient. Lower side 260A may comprise an adhesive 266A. Breathable tape 260T may comprise a backing 211. A first electrode 262A and a second electrode 262B can measure an electrocardiogram signal and treat the patient with a high voltage shock therapy as described above. Adherent patch 260 comprises a third electrode 262C that may comprise a measurement electrode. Adherent patch 260 comprises a fourth electrode 262D that may comprise a measurement electrode. Gel 214A, gel 214B and gel 214C can each be positioned over electrodes 112A, 112B and 112C, respectively. Sensor circuitry as described above can be coupled to the at least four electrodes to measure at least two vectors of the electrocardiogram signal.

A first vector may comprise a horizontal vector 255H that corresponds to a measurement axis extending from electrode 262A to electrode 262B. A second vector may comprise a vertical vector 255V that corresponds measurement axis extending from electrode 262C to electrode 262D. In many embodiments, the vectors of the electrocardiogram signal can be calculated with the processor on the adherent device, for example with known methods of calculating vectors. The first electrode 262A and second electrode 262B can define a line, and the third electrode, for example electrode 262C, comprises an electrode positioned away from the line to measure the at least two vectors of the electrocardiogram signal. In specific embodiments, the at least four electrodes comprise a substantially orthogonal arrangement to measure two substantially orthogonal vectors of the electrocardiogram signal, for example horizontal electrode 265H and vertical electrode 265V. The processor system can be configured to calculate an additional vector of the electrocardiogram signal in response to the at least three vectors, for example with the processor on the adherent device. First electrode 262A and second electrode 262B can be larger than third electrode 262C and fourth electrode 262D, for example at least about twice the diameter and can at least three to four times the diameter.

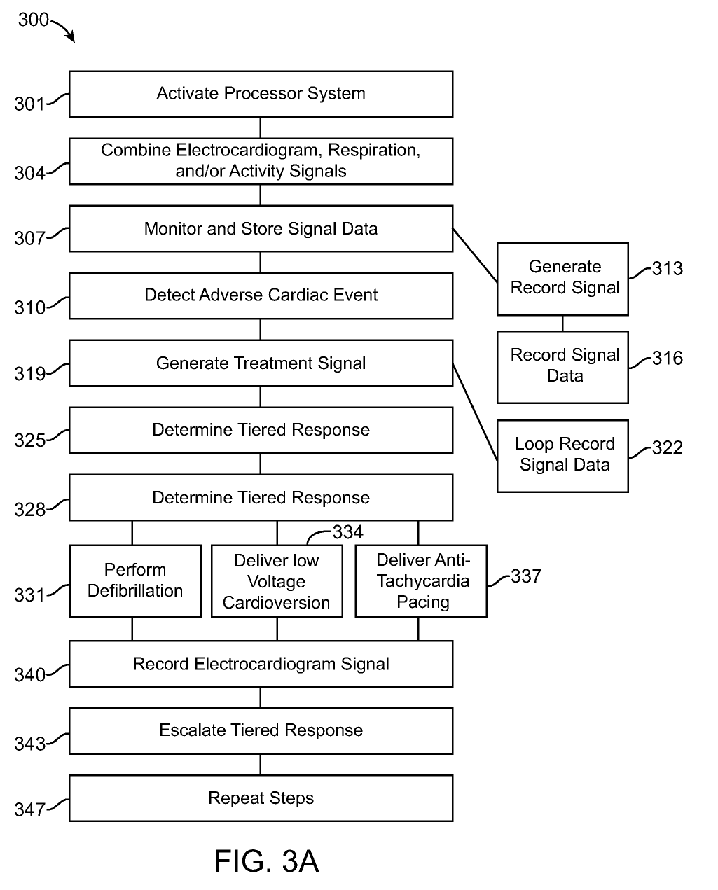

FIG. 3A shows a method 300 for monitoring a patient and treating a patient. A step 301 activates a processor system. A step 304 combines at least two of the electrocardiogram, respiration, and/or activity signals. A step 307 continuously monitors and stores the signal data. In some embodiments, a step may also comprise monitoring a high risk patent post myocardial infarction with the at least two of the electrocardiogram signal, the respiration signal or the activity signal, and/or a bradycardia of the patient at risk for sudden death. The electrocardiogram signal may comprise at least one of a Brugada Syndrome with an ST elevation and a short QT interval or long-QT interval. A step 313 generates a record signal. A step 313 generates a record signal. A step 316 records at least two of the electrocardiogram, respiration, and/or activity signals. A step 310 detects an adverse cardiac event. An adverse cardiac event may comprise an atrial fibrillation in response to the electrocardiogram signal and/or an acute myocardial infarction in response to an ST segment elevation of the electrocardiogram signal. A step 329 generates a treatment signal. A step 322 loop records the signal data. A step 325 generates a treatment signal. A step 328 determines a tiered response. In many embodiments, the tiered response may comprise tiers, or levels, appropriate to the detected status of the patient. A step 331 comprises a first tiered response which performs defibrillation. A step 334 comprises a second tiered response which delivers a low voltage cardioversion. A step 337 comprises a third tiered response which delivers an anti-tachycardia pacing. A step 340 records an electrocardiogram signal. A step 343 escalates the tiered response. A step 347 repeats at lease one of the above steps.

The signals can be combined in many ways. In some embodiments, the signals can be used simultaneously to determine the impending cardiac decompensation.

In some embodiments, the signals can be combined by using the at least two of the electrocardiogram signal, the respiration signal or the activity signal to look up a value in a previously existing array.

TABLE 1
Lookup Table for ECG and Respiration Signals.

| Heart Rate/Respiration | A-B bpm | C-D bpm | E-F bpm |
|---|---|---|---|
| U-V per min | N | N | Y |
| W-X per min | N | Y | Y |
| Y-Z per min | Y | Y | Y |

Table 1 shows combination of the electrocardiogram signal with the respiration signal to look up a value in a pre-existing array. For example, at a heart rate in the range from A to B bpm and a respiration rate in the range from U to V per minute triggers a response of N. In some embodiments, the values in the table may comprise a tier or level of the response, for example four tiers. In specific embodiments, the values of the look up table can be determined in response to empirical data measured for a patient population of at least about 100 patients, for example measurements on about 1000 to 10,000 patients. The look up table shown in Table 1 illustrates the use of a look up table according to one embodiment, and one will recognize that many variables can be combined with a look up table.

In some embodiments, the table may comprise a three or more dimensional look up table, and the look up table may comprises a tier, or level, of the response, for example an alarm.

In some embodiments, the signals may be combined with at least one of adding, subtracting, multiplying, scaling or dividing the at least two of the electrocardiogram signal, the respiration signal or the activity signal. In specific embodiments, the measurement signals can be combined with positive and or negative coefficients determined in response to empirical data measured for a patient population of at least about 100 patients, for example data on about 1000 to 10,000 patients.

In some embodiments, a weighted combination may combine at least two measurement signals to generate an output value according to a formula of the general form

OUTPUT=aX+bY

where a and b comprise positive or negative coefficients determined from empirical data and X, and Z comprise measured signals for the patient, for example at least two of the electrocardiogram signal, the respiration signal or the activity signal. While two coefficients and two variables are shown, the data may be combined with multiplication and/or division. One or more of the variables may be the inverse of a measured variable.

In some embodiments, the ECG signal comprises a heart rate signal that can be divided by the activity signal. Work in relation to embodiments of the present invention suggest that an increase in heart rate with a decrease in activity can indicate an impending decompensation. The signals can be combined to generate an output value with an equation of the general form

OUTPUT=aX/Y+bZ

where X comprise a heart rate signal, Y comprises an activity signal and Z comprises a respiration signal, with each of the coefficients determined in response to empirical data as described above.

In some embodiments, the data may be combined with a tiered combination. While many tiered combinations can be used a tiered combination with three measurement signals can be expressed as

OUTPUT=(ΔX)+(ΔY)+(ΔZ)

where (ΔX), (ΔY), (ΔZ) may comprise change in heart rate signal from baseline, change in respiration signal from baseline and change in activity signal from baseline, and each may have a value of zero or one, based on the values of the signals. For example if the heart rate increase by 10%, (ΔX) can be assigned a value of 1. If respiration increases by 5%, (ΔY) can be assigned a value of 1. If activity decreases below 10% of a baseline value (ΔZ) can be assigned a value of 1. When the output signal is three, a flag may be set to trigger an alarm.

In some embodiments, the data may be combined with a logic gated combination. While many logic gated combinations can be used, a logic gated combination with three measurement signals can be expressed as

OUTPUT=(ΔX) AND (ΔY) AND (ΔZ)

where (ΔX), (ΔY), (ΔZ) may comprise change in heart rate signal from baseline, change in respiration signal from baseline and change in activity signal from baseline, and each may have a value of zero or one, based on the values of the signals. For example if the heart rate increase by 10%, (ΔX) can be assigned a value of 1. If respiration increases by 5%, (ΔY) can be assigned a value of 1. If activity decreases below 10% of a baseline value (ΔZ) can be assigned a value of 1. When each of (ΔX), (ΔY), (ΔZ) is one, the output signal is one, and a flag may be set to trigger an alarm. If any one of (ΔX), (ΔY) or (ΔZ) is zero, the output signal is zero and a flag may be set so as not to trigger an alarm. While a specific example with AND gates has been shown the data can be combined in may ways with known gates for example NAND, NOR, OR, NOT, XOR, XNOR gates. In some embodiments, the gated logic may be embodied in a truth table.

The processor system, as described above, performs the methods 300, including many of the steps described above. It should be appreciated that the specific steps illustrated in FIG. 3A provide a particular method of monitoring and treating a patient, according to an embodiment of the present invention. Other sequences of steps may also be performed according to alternative embodiments. For example, alternative embodiments of the present invention may perform the steps outlined above in a different order. Moreover, the individual steps illustrated in FIG. 3A may include multiple sub-steps that may be performed in various sequences as appropriate to the individual step. Furthermore, additional steps may be added or removed depending on the particular applications. One of ordinary skill in the art would recognize many variations, modifications, and alternatives.

In many embodiments, an adhesive patch to a skin of the patient is adhered to a skin of the patient such that at least two electrodes connected to the patch are electrically patched to the patient. The vectors of the electrocardiogram signal can be calculated with the processor on the adherent device, for example with known methods of calculating vectors. At least two electrodes can define a line, and the third electrode can comprise an electrode positioned away from the line to measure at least two vectors of the electrocardiogram signal. In specific embodiments, at least three electrodes comprise a substantially orthogonal arrangement to measure two substantially orthogonal vectors of the electrocardiogram signal. The processor system can be configured to calculate an additional vector of the electrocardiogram signal in response to the two vectors, for example with the processor on the adherent device.

While the exemplary embodiments have been described in some detail, by way of example and for clarity of understanding, those of skill in the art will recognize that a variety of modifications, adaptations, and changes may be employed. Hence, the scope of the present invention should be limited solely by the appended claims.

What is claimed is:

1. An adherent device to monitor and treat a patient, the device comprising:
an adhesive patch to adhere to a skin of the patient;
at least two electrodes connected to the patch and capable of electrically coupling to the patient;
sensor circuitry coupled to the at least two electrodes and configured to measure at least two of an electrocardiogram signal of the patient, a respiration signal of the patient or an activity signal of the patient;
therapy circuitry coupled to the at least two electrodes and configured to deliver a high-energy shock therapy for cardioversion and/or defibrillation; and
a processor system comprising a tangible medium and coupled to the sensor circuitry and therapy circuitry, the processor configured to generate a treatment signal to deliver the high-energy shock therapy in response to the at least two of the electrocardiogram signal, the respiration signal or the activity signal.

2. The adherent device of claim 1 wherein the adhesive patch comprises a breathable tape affixed to the at least two electrodes and the sensor circuitry and the therapy circuitry are separated from the breathable tape by a gap to allow the tape the breath.

3. The adherent device of claim 1 further comprising isolation circuitry to protect the sensor circuitry from the therapy circuitry when the shock therapy is delivered.

4. The adherent device of claim 3 wherein the isolation circuitry comprises at least one of a capacitor or an electrical switch.

5. The adherent device of claim 1 wherein the processor system comprises a first processor comprising a tangible medium attached to the adherent patch and a second processor comprising a tangible medium at a remote center.

6. The adherent device of claim 1 wherein the processor system is configured to combine the at least two of the electrocardiogram signal, the respiration signal or the activity signal.

7. The adherent device of claim 6 wherein combining comprises the processor system using the at least two of the electrocardiogram signal, the hydration signal, the respiration signal or the activity signal to look up a value in a previously existing array.

8. The adherent device of claim 6 wherein combining comprises at least one of adding, subtracting, multiplying, scaling, or dividing the at least two of the electrocardiogram signal, the respiration signal, or the activity signal.

9. The adherent device of claim 6 wherein the at least two of the electrocardiogram signal, the accelerometer signal, or the respiration signal are combined with at least one of a weighted combination, a tiered combination or a logic gated combination, a time weighted combination or a rate of change.

10. The adherent device of claim 1 wherein the processor system is configured to continuously monitor, store and transmit to a remote center the at least two of the electrocardiogram signal, the respiration signal or the activity signal in response to the treatment signal.

11. The adherent device of claim 1 wherein the processor system is configured to deliver the high-energy therapy and alert a physician in response to an adverse cardiac event.

12. The adherent device of claim 1 wherein the processor system is configured to detect at least one of a T-wave alternans, a pulsus alternans, an autonomic imbalance, a heart rate variability in response to the at least two of the electrocardiogram signal, the respiration signal or the activity signal.

13. The adherent device of claim 1 wherein the processor system is configured to loop record the at least two of the electrocardiogram signal, the respiration signal or the activity signal for diagnosis in response to the treatment signal.

14. The adherent device of claim 13 wherein the processor system is configured to acquire the electrocardiogram signal with a high sampling rate in response to the treatment signal for a period of time before the shock therapy is delivered.

15. The adherent device of claim 1 wherein the processor system is configured to acquire the electrocardiogram signal with a high sampling rate for a period to time in response to the at least two of the electrocardiogram signal, the respiration signal or the activity signal.

16. The adherent device of claim 1 wherein the processor system is configured to detect an event comprising at least one of an atrial fibrillation in response to the electrocardiogram signal or an acute myocardial infarction in response to an ST segment elevation of the electrocardiogram signal.

17. The adherent device of claim 1 wherein the processor system is configured to monitor the electrocardiogram signal and an alert at least one of a remote center, a physician, emergency responder, or family/caregiver when the shock therapy is delivered.

18. The adherent device of claim 1 wherein the processor system is configured to determine a tiered response to the at least two of the electrocardiogram signal, the respiration signal or the activity signal.

19. The adherent device of claim 18 wherein the tiered response comprises a first tier to deliver the shock therapy comprising defibrillation, a second tier to deliver low voltage cardioversion and a third tier to deliver anti-tachycardia pacing.

20. The adherent device of claim 19 wherein the processor system is configured to measure the electrocardiogram signal after the shock therapy is delivered and escalate the therapy to another tier in response to the electrocardiogram signal.

21. The adherent device of claim 1 wherein wireless communication circuitry is configured to transmit the at least two of the electrocardiogram signal, the respiration signal or the activity signal in real time in response to the treatment signal.

22. The adherent device of claim 1 wherein the at least two electrodes comprise at least three electrodes and the sensor circuitry is coupled to the at least three electrodes to measure at least two vectors of the electrocardiogram signal.

23. The adherent device of claim 22 wherein the at least three electrodes comprise at least four electrodes and the sensor circuitry is coupled to the at least four electrodes to measure the at least two vectors of the electrocardiogram signal.

24. The adherent device of claim 22 wherein the at least two electrodes define a line and the least three electrodes comprise an electrode positioned away from the line to measure the at least two vectors of the electrocardiogram signal.

25. The adherent device of claim 22 wherein the at least three electrodes comprise a substantially orthogonal arrangement to measure two substantially orthogonal vectors the electrocardiogram signal.

26. The adherent device of claim 22 wherein the processor system is configured to calculate an additional vector of the electrocardiogram signal in response to the at least two vectors.

27. The adherent device of claim 1 wherein the processor system is configured to generate a record signal to record at least the electrocardiogram signal with high resolution for an arrhythmia log in response to the at least two of the electrocardiogram signal, the respiration signal or the activity signal.

28. The adherent device of claim 27 wherein the processor system is configured to generate the record signal before the treatment in response to the at least two of the electrocardiogram signal, the respiration signal or the activity signal.

29. An method of monitoring and treating a patient, the method comprising:
adhering an adhesive patch to a skin of the patient such that at least two electrodes connected to the patch are electrically coupled to the patient;
measuring at least two of an electrocardiogram signal of the patient, a respiration signal of the patient or an activity signal of the patient with sensor circuitry coupled to the at least two electrodes;
delivering a high-energy shock therapy for cardioversion and/or defibrillation with therapy circuitry coupled to the at least two electrodes; and
generating a treatment signal to deliver the high-energy shock therapy in response to the at least two of the electrocardiogram signal, the respiration signal or the activity signal with a processor system comprising a tangible medium and coupled to the sensor circuitry and therapy circuitry.

30. The method of claim 29 wherein the adhesive patch comprises a breathable tape affixed to the at least two electrodes and the sensor circuitry and the therapy circuitry are separated from the breathable tape by a gap such that the tape the breathes when adhered to the patient.

31. The method of claim 29 further comprising isolating the sensor circuitry from the electrodes and the therapy circuitry with isolation circuitry when the shock therapy is delivered.

32. The method of claim 29 wherein the processor system comprises a first processor comprising a tangible medium attached to the adherent patch and a second processor comprising a tangible medium at a remote center, and the first processor generates the treatment signal with instructions from the second processor.

33. The method of claim 29 wherein the processor system combines the at least two of the electrocardiogram signal, the respiration signal or the activity signal.

34. The method of claim 33 wherein combining comprises the processor system using the at least two of the electrocardiogram signal, the hydration signal, the respiration signal or the activity signal to look up a value in a previously existing array.

35. The method of claim 33 wherein combining comprises at least one of adding, subtracting, multiplying, scaling, or dividing the at least two of the electrocardiogram signal, the respiration signal, or the activity signal.

36. The method of claim 33 wherein the at least two of the electrocardiogram signal, the accelerometer signal, or the respiration signal are combined with at least one of a weighted combination, a tiered combination or a logic gated combination, a time weighted combination or a rate of change.

37. The method of claim 29 wherein the processor system continuously monitors, stores and transmits to a remote center the at least two of the electrocardiogram signal, the respiration signal or the activity signal in response to the treatment signal.

38. The method of claim 29 wherein the processor system delivers the high-energy therapy and alerts a physician in response to an adverse cardiac event.

39. The method of claim 29 wherein the processor system detects at least one of a T-wave alternans, a pulsus alternans, an autonomic imbalance, a heart rate variability in response to the at least two of the electrocardiogram signal, the respiration signal or the activity signal.

40. The method of claim 29 wherein the processor system loop records the at least two of the electrocardiogram signal, the respiration signal or the activity signal for diagnosis in response to the treatment signal.

41. The method of claim 40 wherein the processor system acquires the electrocardiogram signal with a high sampling rate in response to the treatment signal for a period of time before the shock therapy is delivered.

42. The method of claim 29 wherein the processor system acquires the electrocardiogram signal with a high sampling rate for a period to time in response to the at least two of the electrocardiogram signal, the respiration signal or the activity signal.

43. The method of claim 29 wherein the processor system detects an event comprising at least one of an atrial fibrillation in response to the electrocardiogram signal or an acute myocardial infarction in response to an ST segment elevation of the electrocardiogram signal.

44. The method of claim 29 wherein the processor system monitors the electrocardiogram signal and alerts at least one of a remote center, a physician, emergency responder, or family/caregiver when the shock therapy is delivered.

45. The method of claim 29 wherein the processor system determines a tiered response to the at least two of the electrocardiogram signal, the respiration signal or the activity signal.

46. The method of claim 45 wherein the tiered response comprises a first tier to deliver the shock therapy comprising defibrillation, a second tier to deliver low voltage cardioversion and a third tier to deliver anti-tachycardia pacing.

47. The method of claim 46 wherein the electrocardiogram signal is recorded after the shock therapy is delivered and processor escalates the therapy to another tier in response to the electrocardiogram signal.

48. The method of claim 29 wherein wireless communication circuitry transmits the at least two of the electrocardiogram signal, the respiration signal or the activity signal in real time in response to the treatment signal.

49. The method of claim 29 wherein the at least two electrodes comprise at least three electrodes and the sensor circuitry is coupled to the at least three electrodes to measure at least two vectors of the electrocardiogram signal.

50. The method of claim 49 wherein the at least three electrodes comprise at least four electrodes and the sensor circuitry is coupled to the at least four electrodes to measure the at least two vectors of the electrocardiogram signal.

51. The method of claim 49 wherein the at least two electrodes define a line and the least three electrodes comprise an electrode positioned away from the line to measure the at least two vectors of the electrocardiogram signal.

52. The method of claim 49 wherein the at least three electrodes comprise a substantially orthogonal arrangement to measure two substantially orthogonal vectors the electrocardiogram signal.

53. The method of claim 49 wherein the processor system calculates an additional vector of the electrocardiogram signal in response to the at least two vectors.

54. The method of claim 29 wherein the processor system generates a record signal in response to the at least two of the electrocardiogram signal, the respiration signal or the activity signal.

55. The method of claim 54 wherein the processor records the at least two at least the electrocardiogram signal, the respiration signal of the patient or the activity signal of the patient with high resolution for an arrhythmia log in response to the record signal.

56. The method of claim 29 wherein the processor system generates a record signal to record at least the electrocardiogram signal with high resolution for an arrhythmia log in response to the at least two of the electrocardiogram signal, the respiration signal or the activity signal.

57. The method of claim 56 wherein the processor system generates the record signal before the treatment in response to the at least two of the electrocardiogram signal, the respiration signal or the activity signal.
